(19) United States
(12) Patent Application Publication
(10) Pub. No.: US 2025/0312067 A1
(43) Pub. Date: Oct. 9, 2025

(54) DEVICES AND METHODS FOR TREATING SHOULDER DYSTOCIA

(71) Applicant: FetalEase, Zichron Yaacov (IL)

(72) Inventors: Abraham J. Yaari, Zichron Yaacov (IL); Aaron Feldman, Zichron Yaacov (IL)

(73) Assignee: FetalEase, Zichron Yaacov (IL)

(21) Appl. No.: 19/100,605

(22) PCT Filed: Aug. 7, 2023
(86) PCT No.: PCT/US2023/029587

§ 371 Date: Feb. 3, 2025

Related U.S. Application Data
(60) Provisional application No. 63/399,253, filed on Aug. 19, 2022.
(60) Provisional application No. 63/399,255, filed on Aug. 19, 2022.

(51) Int. Cl. A61B 17/44; A61B 17/00; A61B 17/28; A61M 25/10

(52) U.S. Cl. CPC A61B 17/44; A61B 17/2833; A61B 2017/00557; A61B 2017/00849; A61M 25/1011

(57) ABSTRACT

A medical device for treating shoulder dystocia having a first arm having a proximal portion, a distal portion and a first curved surface, a second arm having a proximal portion, a distal portion and a second curved surface and a shoulder engagement section positioned at least between the distal portion of the first and second arms and connecting the first and second arms. The engagement section is configured to contact and press down on the baby to reposition the baby.

This application claims priority to provisional application Ser. No. 63/399,253, filed on Aug. 19, 2022, and provisional application Ser. No. 63/399,255, filed on Aug. 19, 2022. The entire contents of each of these applications are incorporated herein by reference.

### BACKGROUND

### Technical Field

This application relates to a device to assist the physician/clinician/birth attendant in delivery of a baby and more particularly to a device for delivery of the fetal shoulder in the case of shoulder dystocia.

### Background of Related Art

Shoulder dystocia is a condition whereby after delivery of the head of the baby, the anterior shoulder of the baby cannot pass below the pubic symphysis. This creates an obstetric emergency as the baby's life is endangered if not delivered because of compression of the umbilical cord within the birth canal. Additionally, shoulder dystocia can cause several types of injury to the baby as a result of the forces of labor, e.g., maternal pushing acting on the baby, the physician's pulling on the fetal head or the physician's maneuvers undertaken to free the shoulder for delivery. There are various maneuvers currently being utilized to handle shoulder dystocia, with varying degrees of success and varying risk of causing temporary or permanent injury.

The incidence of shoulder dystocia is increasing as a result of the increase in the average birth weight of babies. The increase in birth weight is due in part to the rise in adult obesity and gestational diabetes as well as the increase in the caloric intake during pregnancy.

Consequently, as the size of babies continues to increase, the incidence of shoulder dystocia has likewise been increasing. Therefore, the need exists to improve delivery of the baby in cases of, or anticipated cases of, shoulder dystocia to reduce the chances of injuring the baby and reduce the incidences of associated morbidity and mortality.

### SUMMARY

U.S. Pat. No. 9,474,549 (hereinafter the '549 patent) discloses devices for effectively treating shoulder dystocia. The inventor of the '549 patent conceived of unique improvements and enhancements to the devices disclosed in the '549 patent which can in certain clinical situations provide advantages and increase the effectiveness and/or ease of use of the device. These variations of the device are each described in detail below.

The inventor of the 9,474,549 patent also conceived of alternative devices for treating shoulder dystocia which can in certain clinical situations provide advantages and increase the effectiveness and/or ease of use. These devices provide alternatives to the engagement member of the '549 patent and to the engagement member improvements disclosed herein. These alternative devices are also described in detail below.

The devices and methods of the present invention provide for delivery of a baby in the case of shoulder dystocia. In general, the device is inserted into the pelvis below the pubic symphysis and manipulated by the physician to free the baby's shoulder from under the pubic symphysis to allow delivery. The present invention also provides a method of freeing the baby's shoulder to deliver the baby in the case of shoulder dystocia. The devices and methods of the present invention can also be used to prevent the entrapment of the fetal shoulder before it occurs if such an event is anticipated. Various embodiments of the devices of the present invention are disclosed in detail below.

The devices of the present invention in certain applications can be inserted below the posterior shoulder of the baby to allow rotation of the baby.

In accordance with one aspect of the present invention, a medical device for treating shoulder dystocia is provided comprising a first arm having a proximal portion, a distal portion and a first curved surface, and a second arm having a proximal portion, a distal portion and a second curved surface. A shoulder engagement section is positioned at least between the distal portion of the first and second arms and connects the first and second arms, the engagement section having an upper surface and a lower surface having an atraumatic surface and configured to contact and press down on a baby to reposition the baby upon manipulation of the first and second arms. A spacer extends from the upper surface of the engagement section.

In some embodiments, the spacer comprises an inflatable balloon. The device can include a pump and a tube fluidly connected to the balloon, the pump attached to one of more of the first arm, second arm or engagement member.

In some embodiments, the spacer comprises a second balloon positioned adjacent the balloon. The balloons can be independently inflatable. In other embodiments, the balloon has a first compartment and a second separate compartment, the first and second compartments independently inflatable to selectively space regions of the engagement member.

In accordance with another aspect of the present invention, a medical device for treating shoulder dystocia is provided comprising a first arm having a proximal portion, a distal portion and a first curved surface, and a second arm having a proximal portion, a distal portion and a second curved surface. A shoulder engagement section is positioned at least between the distal portion of the first and second arms and connects the first and second arms, the engagement section having an upper surface and a lower surface having an atraumatic surface and configured to contact and press down on a baby to reposition the baby upon manipulation of the first and second arms. The upper surface has a concave section forming a depression to accommodate the shoulder of the baby.

In some embodiments, the concave section is formed in a central region of the engagement member. In some embodiments, a second concave section is positioned in the upper surface of the engagement member.

In accordance with another aspect of the present invention, a medical device for treating shoulder dystocia is provided comprising a first arm having a proximal portion, a distal portion and a first curved surface, and a second arm having a proximal portion, a distal portion and a second curved surface. A shoulder engagement section is positioned at least between the distal portion of the first and second arms and connects the first and second arms, the engagement section having an upper surface and a lower surface having an atraumatic surface and configured to contact and press down on a baby to reposition the baby upon manipulation of the first and second arms. A locking mechanism is engageable with the first and second arms to retain the first and second arms in position during use.

In some embodiments, the locking mechanism comprises a projection and groove engagement such that when the first and second arms are brought together, the projection and groove interlock.

In some embodiments, the locking mechanism has a plurality of teeth engageable by a pawl to selectively lock the arm in a number of desired positions.

In accordance with another aspect of the present invention, a medical device for treating shoulder dystocia is provided comprising a first arm having a proximal portion, a distal portion and a first curved surface, and a second arm having a proximal portion, a distal portion and a second curved surface. A shoulder engagement section is positioned at least between the distal portion of the first and second arms and connects the first and second arms, the engagement section having an upper surface and a lower surface having an atraumatic surface and configured to contact and press down on a baby to reposition the baby upon manipulation of the first and second arms. A lubricant is dispersible on the engagement member.

In some embodiments, the lubricant is contained in a lubricant bag positioned adjacent the engagement member. In some embodiments, the lubricant encapsulates the engagement member. In some embodiments, the lubricant is contained in a container connected to a tube having an opening adjacent the engagement member.

In accordance with another aspect of the present invention, a medical device for treating shoulder dystocia is provided comprising a first arm having a proximal portion, a distal portion and a first curved surface and a second arm having a proximal portion, a distal portion and a second curved surface. A cord is positioned between the distal portion of the first and second arms and connects the first and second arms, the cord configured to contact and press down on a baby to reposition the baby upon manipulation of the first and second arms.

In some embodiments, the first and second arms are movable from a first position wherein the cord is not taut to a second more spread position wherein the cord becomes taut to apply a force to the baby's shoulder.

In accordance with another aspect of the present invention, a medical device for treating shoulder dystocia is provided comprising a first arm having a proximal portion, a distal portion and a first curved surface, and a second arm having a proximal portion, a distal portion and a second curved surface. A balloon is positioned between the distal portion of the first and second arms, the balloon inflatable to contact and press down on a baby to reposition the baby upon manipulation of the first and second arms.

In some embodiments, the device includes a pump and a tube fluidly connected to the balloon, the pump attached to one of more of the first arm, second arm or engagement member.

In some embodiments, the balloon has multiple compartments independently inflatable.

### BRIEF DESCRIPTION OF THE DRAWINGS

Preferred embodiment(s) of the present disclosure are described herein with reference to the drawings wherein:

FIG. 1A is a perspective view of a first embodiment of the device of the present invention having a spacer balloon on the engagement member, the handles shown in the open (spread) position and the balloon shown in the non-inflated position;

FIG. 1B is a perspective view of an alternate embodiment of the device of the present invention having a pair of spacer balloons (shown in the non-inflated condition), the handles shown in the open position;

FIG. 1C is a side view of an alternate embodiment of the arm of the device of FIG. 1A;

FIG. 1D is a side view of another alternate embodiment of the arm of the device of FIG. 1A;

FIG. 2 is a perspective view of an alternate embodiment of the device of the present invention having a balloon pump in the handle, the handles shown in the open (spread) position and the balloon shown in the non-inflated position;

FIG. 3A is a perspective view of an alternate embodiment of the device of the present invention having a concavity in the engagement area, the handles shown in the spaced position;

FIG. 3B is a perspective view of an alternate embodiment of the device of the present invention having two concave regions in the engagement member;

FIG. 4A is a perspective view of an alternate embodiment of the device of the present invention having a locking mechanism to fix the handles to each other, the handles shown in the spaced position;

FIG. 4B is a close up view of a section of the locking mechanism of FIG. 4A;

FIG. 4C is a close up view of an alternate embodiment of the locking mechanism for the handles;

FIG. 4D is a close up view of an alternate embodiment of the locking mechanism for the handles;

FIG. 5 is a perspective view of an alternate embodiment of the device of the present invention having a bifurcated handle;

FIG. 6 is a perspective view of an alternate embodiment of the device of the present invention having a lubricant sac positioned over the engagement member;

FIG. 7 is a perspective view of an alternate embodiment of the device of the present invention having a lubricant pump;

FIG. 8A is a perspective view of an alternate embodiment of the device of the present invention having a straight arm;

FIG. 8B is a perspective view of an alternate positioning/use of the device of the present invention having a curved arms coming in from below;

FIG. 9 is a perspective view of the device of FIG. 1A shown in use in contact with the shoulder of a baby; and

FIGS. 10-12 illustrate alternate embodiments of the devices of the present invention which do not have the spreadable engagement member as in FIGS. 1-8 wherein:

FIG. 10 is a perspective view of a first embodiment of the device of the present invention having a cord between the handles, the handles shown in the open (spread) position and the cord in the taut position;

FIG. 11 is a perspective view of an alternate embodiment of the device of the present invention having a balloon supported by a pair of arms; and

FIG. 12 is a perspective view of an alternate embodiment of the device of the present invention having a balloon supported by a pair of arms.

### DETAILED DESCRIPTION OF PREFERRED EMBODIMENTS

Referring now in detail to the drawings wherein like reference numerals identify similar or like components throughout the several views, various embodiments of the delivery device of the present invention are illustrated. The devices are designed to deliver a baby in cases of anticipated or actual shoulder dystocia. As described above, in cases of shoulder dystopia, the baby's head is delivered but the shoulder is engaged with the pubic arch, thus blocking delivery. The devices of the present invention, as described in detail below, are placed in the birth canal, under the pubic arch and into contact with the shoulder of the baby. In some embodiments, the device has an engagement member conforming to the baby's anatomy.

Once in position, e.g., adjacent or in contact with the shoulder, the physician applies a downward force on the device causing the shoulder contacting (engaging) component of the device to atraumatically press against the baby's shoulder, thereby releasing the shoulder from the pubic arch so the baby can be safely delivered.

In some embodiments, once in position, e.g., adjacent or in in contact with the shoulder, the arms of the device are manipulated, e.g., opened to a more spread position, rotated and/or pivoted. Once manipulated, the physician applies the downward force (by moving the arms forward to the maternal naval) on the arms of the device causing the shoulder contacting (engaging) material or component of the device to atraumatically press against the baby's shoulder, thereby releasing the shoulder from the pubic arch so the baby can be safely delivered.

Note as used herein the term “distal” denotes the portion or section of the device further from the user and the term “proximal” denotes the portion or section of the device closer to the user. “Lower surface” denotes the surface in contact with the baby.

### Engagement Member Improvements and Enhancements

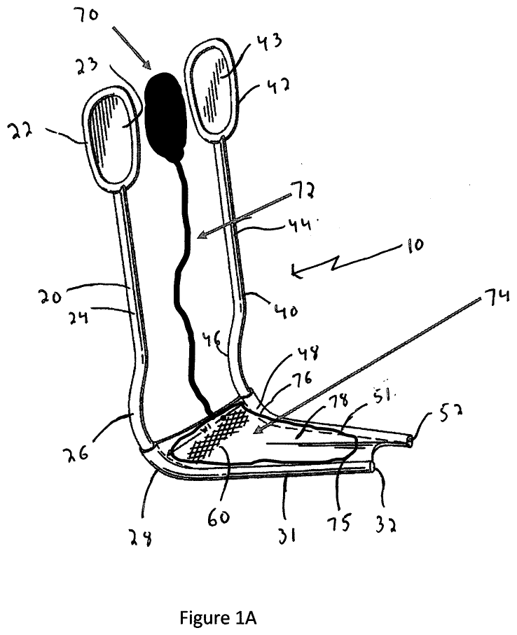

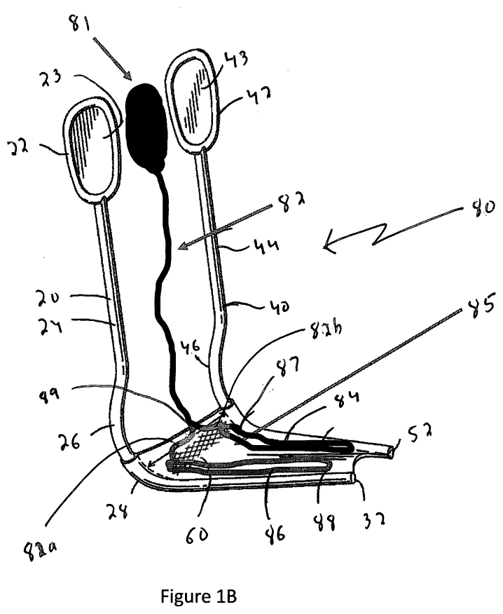

FIG. 1A-8B illustrate embodiments providing enhancements to the devices of the '549 patent.

Turning initially to the first embodiment of the device and with initial reference to FIG. 1A, the device of the present invention for treating shoulder dystocia is designated generally by reference numeral 10 and includes a first arm 20, a second arm 40 and an engagement member 60 spanning the two arms 20, 40 at a distal region. Arms 20 and 40 in a preferred embodiment are substantially identical, although they could be varied from each other as long as their function as described herein is maintained. Arm 20 has a grasping handle 22 at a proximal end, a substantially straight or substantially linear arm portion 24, a first curve 26, a curved portion 28, and a substantially straight or substantially linear arm portion 31 terminating in a distal tip 32. The arm 40 similarly has grasping handle 42 at a proximal end, a substantially straight or substantially linear arm portion 44, a first curve 46, a curved portion 48, and a substantially straight or substantially linear arm portion 51 terminating in a distal tip 52. The distal tips 32, 52 of arms 20, 40 are preferably round to provide an atraumatic blunt end. The arms of this embodiment as well as the other embodiments disclosed herein can be composed of a metal or plastic material.

Note in an alternate embodiment, cach arm can have an upward curve instead of a substantially linear portion as shown in the embodiment of FIG. 1C. More specifically, each arm 20a (only one of which is shown in FIG. 1C) has a substantially straight or substantially linear arm portion 24a, a first curve 26a, a curved portion 28a, and a curved arm portion 31a (instead of a linear arm portion) terminating in an atraumatic distal tip 32a. Arm 20 could also have a grasping handle 22a like handle 23 of FIG. 1A.

Note in an alternate embodiment, cach arm can have a S-curve instead of the substantially linear portion 31, 51 as shown in the alternate embodiment of FIG. 1D. More specifically, cach arm 20b (only one of which is shown in FIG. 1C) has a grasping handle 22b, a substantially straight or substantially linear arm portion 22b, a first curve 26b, a curved portion 28b, and a S-curved arm portion 31b terminating in an atraumatic distal tip 32b. This configuration provides the arm with a double curve. Consequently, in this embodiment, the curve would conform to the contour of the baby's neck, and the lower aspect of the pubic bone. However, once in position, the arms would be opened in the same manner as in the embodiment of FIG. 1A, and the handles manipulated in the same manner as in FIG. 1A, to press the engagement component against the baby's shoulder to provide a force in a direction away from the pubic arch to release the baby's shoulder.

The device of FIG. 1A can be inserted starting along the back of the baby and then moved to along the shoulder, or alternatively, due to the single curve, can be initially inserted over the fetal shoulder and under the pubic bone. This straighter arm version in some applications can be easier to slide into the pelvic area under the pubic arch. Also, this straighter version can better accommodate different pelvic shapes in certain applications.

Referring back to FIG. 1A, the grasping handles 22 and 42 preferably have solid surfaces 23, 43, respectively, for engagement by the physician's thumbs to provide leverage for a downward movement of the device 10 as described below in conjunction with the method or use. The grasping handles 22 and 42 can alternatively be grasped by other fingers or held in the palm of the physician's hand as alternate ways to provide the appropriate leverage to apply the necessary force to dislodge the baby's shoulder from engagement with the pubic arch as described below. The grasping handles 22, 42 (as well as the handles of the other embodiments disclosed herein) can be composed of a metal or plastic material.

An engagement component (also referred to herein as an engagement section or engagement member) of the device 10 is designated by reference numeral 60 and spans a distal region between the two arms 20, 40 at the linear portions 31 and 51. The space between the two arms preferably ranges from about 4 cm to about 10 cm, and preferably between about 5 cm and about 8 cm, although other distances are also contemplated. The engagement section in the open (unfolded/spread) position preferably has a length of about 8 cm to about 12 cm and a width of about 4 cm to about 10 cm, and preferably between about 5 cm and about 8 cm, although other dimensions are also contemplated. The engagement section 60 in some embodiments has a more flexible proximal section to provide greater elasticity for conformance to the baby's shoulder. The distal section of the engagement member can more rigid, and in some embodiments can be non-elastic, to apply a sufficient force against the baby's shoulder when a downward force is applied by manipulation of the arms 20, 40. In some embodiments, the more flexible proximal section 62 occupies less than 50 percent of the total engagement section area, although it could occupy more or less of the total engagement section area. The different rigidity can be achieved by different materials, different thicknesses, different material properties and/or different widths of the engagement member.

The engagement section can in some embodiments comprise a material in the form of a sheet or a cloth or a plastic material, and can have a reinforcement harder material attached thereto or positioned therein to rigidify the distal section or two separate materials (or materials of differing hardness) can form the engagement component, with the material forming the distal section more rigid and the material forming the proximal section more flexible and stretchable. The two sections of different materials can be attached by various known methods such as being sewed together. The less elastic or non-elastic distal region provides a strong surface for manipulation of the baby in the methods described below.

The engagement section or component can in some embodiments be formed in a substantially trapezoidal shape, with a shorter width at the distal end than the proximal end and providing a more rigid and less stretchable distal section.

With continued reference to FIG. 1A, the device 10 includes a spacer on an upper surface of the engagement member 60. The spacer in this embodiment is in the form of a balloon, although other spacers atop the engagement member to perform the function described herein are also contemplated. Balloon 74 is positioned atop the engagement member 60 and acts as a spacer. That is, if one side of the engagement element 60 forms a closed space, then the inflation of this space after positioning the device correctly over the shoulder will push the shoulder out of the trap behind the pubic bone to the other side (to the oblique diameter). The balloon, if dimensioned to cover a large area of the engagement member 60, can push the baby downwards. In some instances, the inflation of the balloon 60 can provide a sufficient force on the shoulder to save the clinician from having to do the forward and lateral movement of the device. In other instances, the user may need to manipulate the handles to provide a further downward force to free the shoulder. The balloon 74 is shown in the deflated/non-inflated state/condition in FIG. 1A.

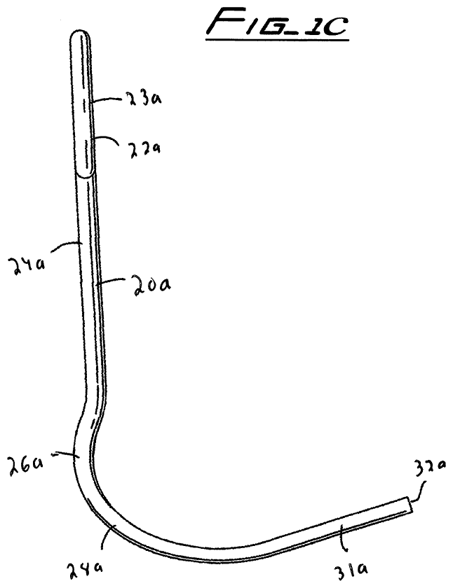

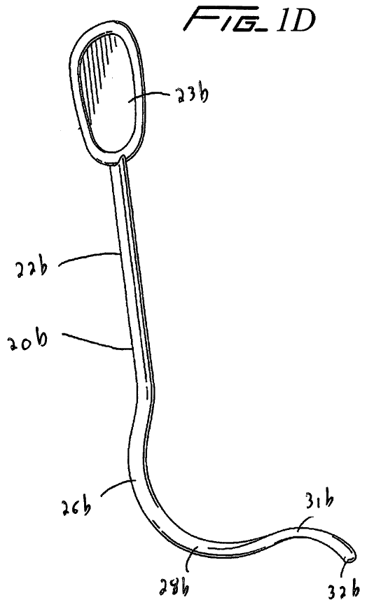

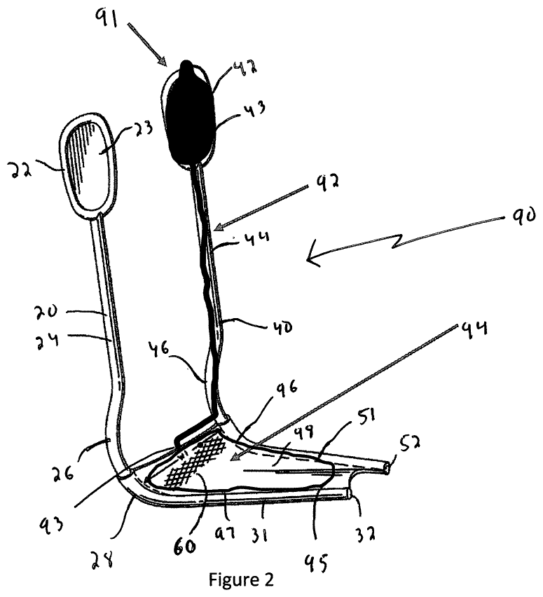

As shown in FIG. 1A, the balloon 74 can be inflated by a pump 70 connected to a tube 72 which is fluidly connected to an interior of the balloon 74. The pump 70 can be unattached to the handles 22, 42 as shown or alternatively the pump 70 can be attached to one of the handles 22, 42 or arms 20, 40 and/or the tube 72 can be attached to one of the handles 22, 42 or arms 20, 40. This is shown for example in the embodiment of FIG. 2 wherein the balloon 94 of device 90 is inflated via a pump 91 attached to handle 42 (or alternatively attached to handle 22) and the tube 92 is attached to arm 40 (or alternatively arm 20). As shown, the tube 72 is attached to the proximal end of balloon 74 so its opening is in communication with the interior of the balloon 94 for insertion of inflation fluid. Balloon 94 has a central region 98, side ends 96, 97, a distal end 95 and a proximal end 93. The balloon 94 is shown in the deflated/non-inflated state/condition in FIG. 2. Except for the position of the pump 91 and tube 92, device 90 is the same in configuration and function as device 20 of FIG. 1A.

FIGS. 1A (and 2) depict a single balloon 74 (94), however in alternate embodiments, multiple balloons can be provided. For example, in the embodiment of FIG. 1B, inflatable section 85 of device 80 has two spaced apart balloons 84, 86. Like balloon 74 of FIG. 1A, balloons 84, 86 are positioned on a top (upper) surface of the engagement member 60 and inflated by air flow via pump 81 and tube 82. The tube 82 can bifurcate into two tubes such that tube 82a is in fluid communication with the interior of balloon 86 and tube 82b is in fluid communication with the interior of balloon 84. A single pump 81 can be used to inflate the balloons 84, 86. The balloons 84, 86 can be inflated simultaneously or alternatively inflated individually/independently such as by provision of a valve or switching mechanism 89 at the bifurcation to direct inflation fluid to the selected balloon 84, 86. In alternate embodiments, a separate pump can be provided for cach balloon to inflate the balloons 84, 86 individually/independently. Individual inflation can direct the movement of the baby's shoulder on a selected side. Alternatively, a single balloon with separate inflatable compartments can be provided to inflate select regions to engage the baby's shoulder. There can be two compartments or alternatively more than two compartments to create multiple compartments to selectively inflate various balloon regions to enable selective spacing of the engagement member.

The balloons of FIGS. 1A, 1B and 2 are shown in the flat, non-inflated state/condition and can be inflated to various pressure/sizes as selected by the user via the hand pump. Automatic pumps for inflating the balloons are also contemplated.

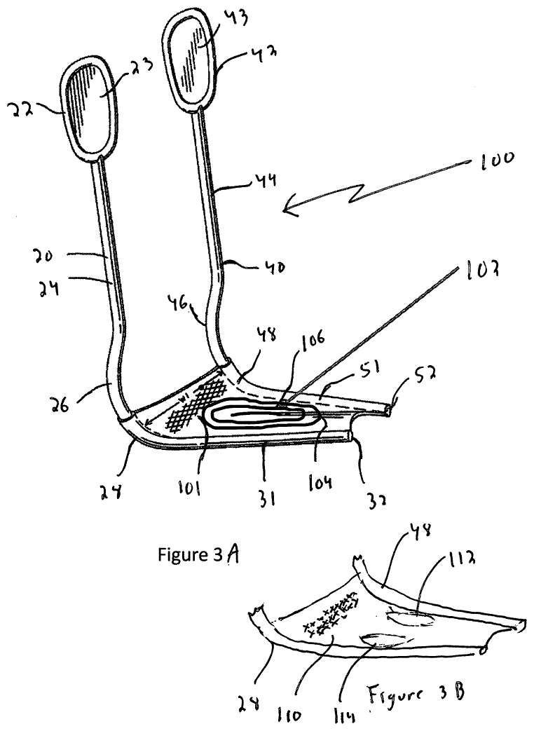

FIG. 3A depicts an alternative embodiment of the device of the present invention. The arms and handles of the device are the same as in FIG. 1A. Device 100 differs from device 10 in that it does not have an inflatable balloon. It also differs from device 10 in that the engagement member 101 has a depression or concave section 102, defined by a concavity 106 in an upper surface. The concavity 106 is formed in a central region of the engagement member 101 as shown, with distal end 104 of the concavity spaced proximally from the distal end of the engagement member 101. In all other respects the engagement member, e.g., materials, rigid sections, etc., and its various alternatives/options are the same as described herein.

More than one concavity can be provided such as in the embodiment of FIG. 3B wherein engagement member 110 has two side by side spaced apart concavities 112, 114.

The concave section(s) in the engagement element can help trap/grab the baby's shoulder. It can accommodate the baby's shoulder and make it easier to turn the shoulder to the side.

The concavity (or concavities) can be oval shaped, circular or of other configurations or sizes and located in the engagement section in regions other than that shown. Also, although shown on the upper surface, in alternate embodiments, the concave section/sections can be on the lower surface of the engagement member.

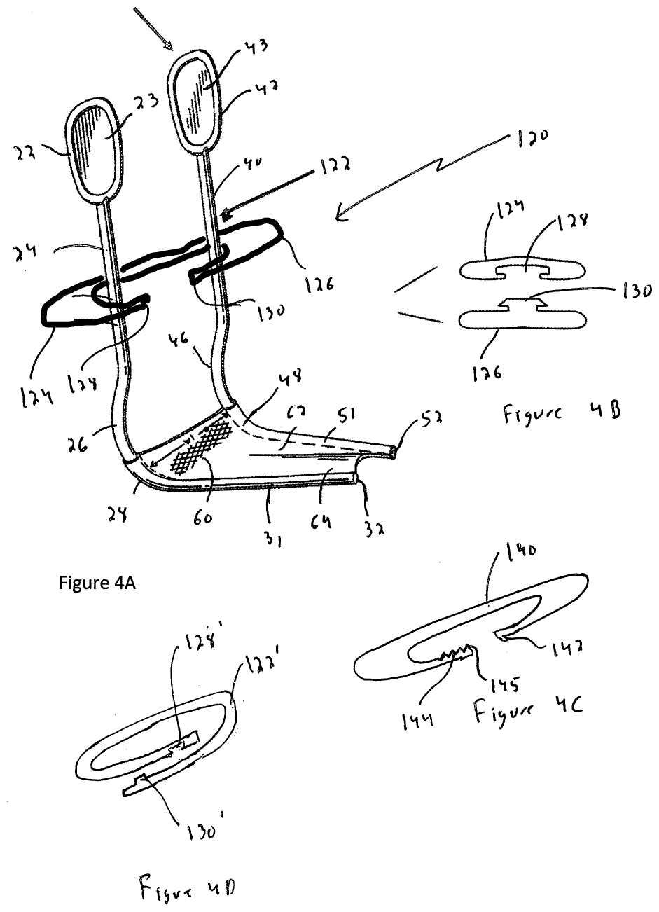

In some embodiments, the handles of the devices disclosed herein are configured to lock (interlock) together. This is shown for example in FIGS. 4A-4C which show device 10 by way of example. However, it should be understood that the locking features/mechanisms disclosed herein could be used with the other devices disclosed herein.

As shown, the arms 20, 40 of the device 120 of FIG. 4A are identical to the arms of device 10, the only difference being the locking mechanism 122. The locking mechanism 122 is designed to retain the handles 22, 42 in position with respect to each other during use. That is, in use, the physician separates the grasping handles 22, 42 to spread the engagement section 60 from the closed position to the open spread position, thereby enveloping a portion of the baby's shoulder. The handles 22, 42 can be locked in the open position. (Alternatively or additionally they can be locked in the closed position). Then, the physician presses down on grasping handles 22, 42, utilizing solid surfaces 23, 43 of grasping handles 22, 42 for leverage to pivot the proximal portion of the arms 20, 40 downwardly to force engagement section 60 downwardly. This force pushes the baby away from the pubic arch, thereby freeing the baby's shoulder from the pubic arch for delivery of the baby. Thus, the baby's shoulder is released from the arch in a quick and atraumatic fashion. The locking of the handles 22, 42 facilitates the use/manipulation of the device when applying downward pressure as the handles 22, 42 are not movable relative to cach other.

In certain applications, in addition to the downward force, the handles 22, 42 can be rotated to the side (toward the fetal chest) or toward the baby's back to move the baby's shoulder to an oblique diameter of the pelvis. That is, such pivoting movement of the handles 22, can rotate the baby, e.g., rotate the baby's shoulder, about 45 degrees, gaining two advantages: 1) the anterior shoulder is not hindered by the pubic arch; and 2) the oblique diameter of the pelvic outlet is larger than the anterior-posterior diameter of the mother, and therefore it is easier for the baby to come out through this position aided by pushing of the mother. The locking of the handles 22, 42 can facilitate such rotational/pivoting movement of the device as the handles 22, 42 are not movable relative to each other.

In the embodiment of FIGS. 4A and 4B, the locking mechanism 122 includes a locking structure/device having a projection and groove engagement. Side 126 of C-shaped locking device has a projection 130 extending in a direction toward the opposing side 124. The projection 130 is configured to fit, e.g., snap into, groove or slot 128 of opposing side 124 of locking mechanism for a snap fit or frictional engagement of the two ends. The locking device 122 is shown in the non-locking position in FIG. 4A. When the handles 22, 42 (and respective arms 24, 40) are brought together in a more closed position, the projection 130 and groove 128 are likewise brought toward cach to engage to lock the arms 24, 40 (and respective handles 22, 42) in this position. In alternate embodiments, the arms 24, 40/handles 24, 42 are locked when they are spread to a more open position. In such embodiments, the locking mechanism would be engaged in the handle position of FIG. 4A and released to move the handles 22, 42 closer. This can be achieved for example in the embodiment of FIG. 4D wherein projection 130′ and groove 128′ are on the sides of the locking device 122′ and when the arms 24, 40 open (spread) come into engagement to interlock.

The locking mechanism 122 can be configured to lock the arms 24, 40 in a predetermined position, i.e., a predetermined distance from each other, depending on the inward extension of the projection and/or groove. The locking mechanism is shown symmetric and C-shape, however, it could also be asymmetric so one end side 124 or 126 extends more inwardly than the opposing end, but still performs the aforedescribed interlocking function. The locking mechanism 122 is shown at an intermediate portion of the arms 24, 40 but alternatively can be closer or further from handles 22, 42 than the position illustrated.

In alternate embodiments, the arms/handles can be locked in various positions rather than in a single position of FIG. 4A. FIG. 4C shows such locking mechanism by way of example. In the embodiment of FIG. 4C, the locking device 140 has a rack and pawl engagement which enables the arms/handles to be locked in a variety of clinician selected positions. That is, the pawl/protrusion 142 is engageable with any of the teeth 144 along the rack when the handles are moved with respect to each other (opened or closed) as the protrusion 142 and teeth 145 engage to retain the handles. As can be appreciated, if the handles 24, 40 are moved toward or away from each other, the protrusion 142 will engage another tooth 144 of rack 145 thus enabling the handles 24, 40 to be locked in a variety of selected positions. The locking mechanism 140 is shown symmetric and C-shape, however, it could also be asymmetric so one end extends more inwardly than the opposing end, but still performs the aforedescribed interlocking function. The locking device 140 can be positioned at an intermediate portion of the arms 24, 40 as in locking mechanism 122 of FIG. 4A, or can be closer or further from handles 22, 42.

It should be appreciated that other locking mechanisms to achieve the above-described single position or multiple position handle locking are contemplated including clasps, frictional engagement, snap locks, etc.

In some embodiments, the front of one or both of the arms could have multiple protrusions and the back of one or both arms have multiple depressions so that when the arms are crossed and pressed together, they interlock due to the protrusion/depression engagement, thereby locking the arms/handles in position.

It should be appreciated that the handle locking mechanisms can be used with any of the embodiments of the device disclosed herein.

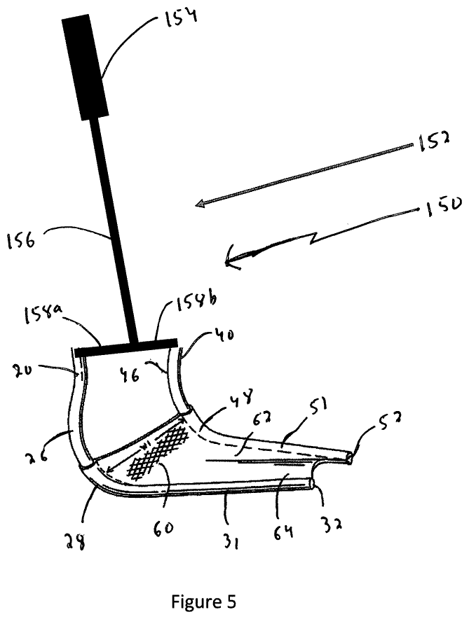

In the embodiment of FIG. 5, device 150 has a bifurcated handle 152. Handle 152 has a grip portion 154 and a shaft portion 156 which bifurcates into left and right shafts 158a, 158b which are either integral with or attached to arms 20, 40 adjacent respective curved portions 26, 46 or at other portions. Such bifurcated handle 152 can be provided in other embodiments of the device. The shafts 158a, 158b can be rigidly attached to the arms 20, 40 so there is no movement of the arms; alternatively, they can be attached to effect or enable movement of the arms away and/or toward each other, e.g., the shafts 158a, 158b can pivot, bend, flex, fold, etc. with respect to the shaft portion 156.

In some embodiments, lubricant is provided on the engagement member. This can be achieved via a lubricant bag or a lubricant pump, both of which are discussed in detail below.

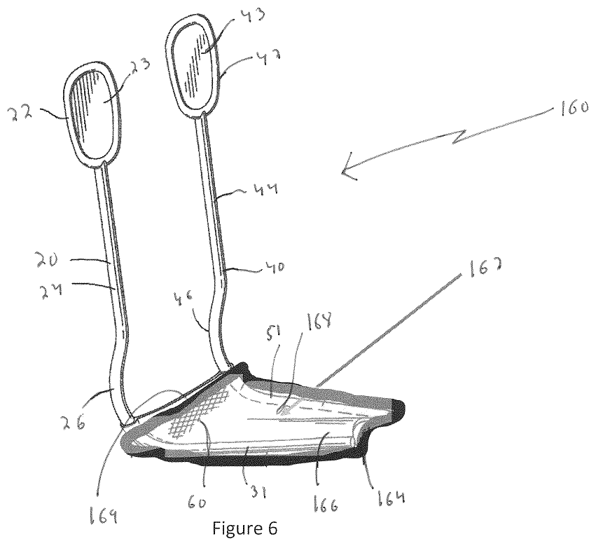

In the embodiment of FIG. 6, the engagement member 60 and distal portions of the arms 31, 51 of device 160 are the same as device 10 of FIG. 1 except the engagement member 60 is encased within a lubricant bag or sac 162. The lubricant bag 162 as shown extends from proximal end 169 to end 164 at a distal end, fully encapsulating the engagement member 60. In alternate embodiments, the bag 162 could cover less than the entire region of the engagement member 60, e.g., only at the distal end of the device. The bag 162 can be torn away to release the lubricant just before use. Provision of the lubricant bag 162 could eliminate the need for the clinician to put gel on the device prior to use which is time consuming and makes the clinician's fingers slippery.

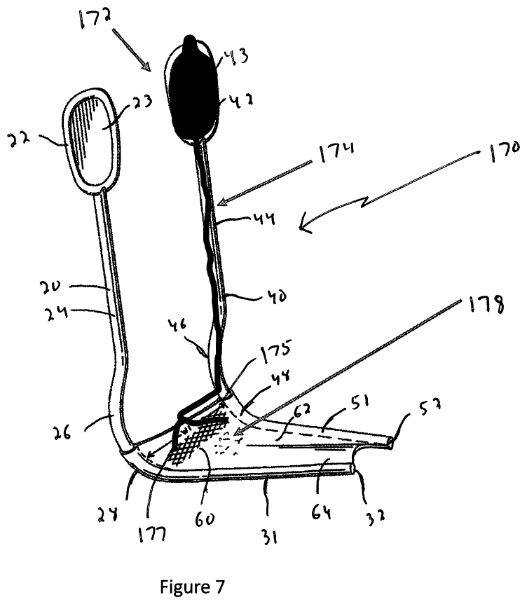

As an alternative to the lubricant sac, the device can include a lubricant container attached to one of the handles. When the container is squeezed, the lubricant in the container is ejected through a short tube or tubes onto the engagement member. This is shown in the alternate embodiment of FIG. 7. Device 170 has a pump 172 which provides a container for the lubricant. The pump 172 is connected to the grasping handle 42 (or alternatively to handle 22). A tube 174 extends alongside handle 40 (or alternatively handle 24) and terminates adjacent the engagement member 60. The tube 174 can have an opening or multiple openings at its distal end for exit of the lubricant onto the engagement member 60. Alternatively, the tube 174 can split into two tubes 175, 177, as illustrated, each having a distal opening, to improve the dispersion of the lubricant 178 on the engagement member 60. Multiple openings, such as side openings in the wall of the tube, can be provided in the tubes 175, 177 to provide further dispersion. Additional tubes could also be provided to provide further lubricant dispersion. The tube 174 is shown alongside the handle 40, and it is also contemplated that the tube 174 can alternatively be positioned within the handle 40 of the device. In use, the pump/container 172 can be squeezed to release the lubricant through the lumen/channel of the tube 174 and onto the engagement member 60. Other ways to disperse the lubricant are also contemplated. Additionally, the lubricant container could be a separate unit unattached or remote from the device to supply lubricant to one or more tubes connected to the device.

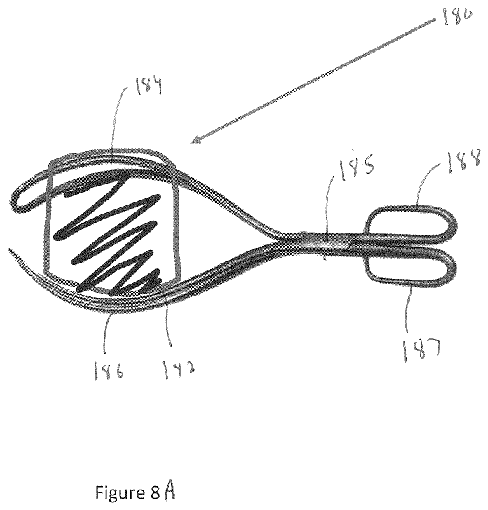

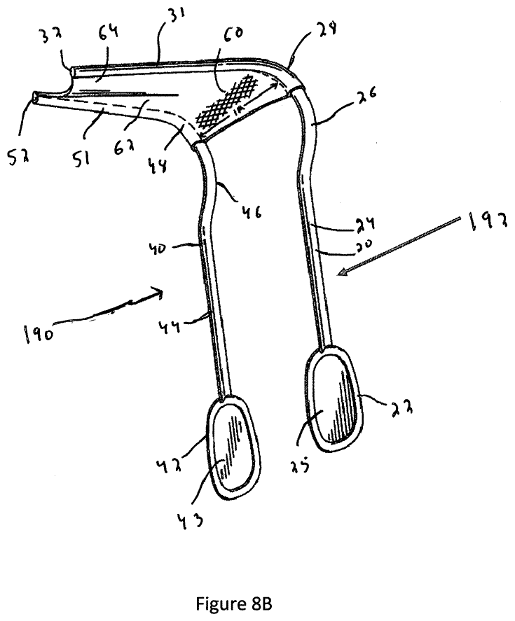

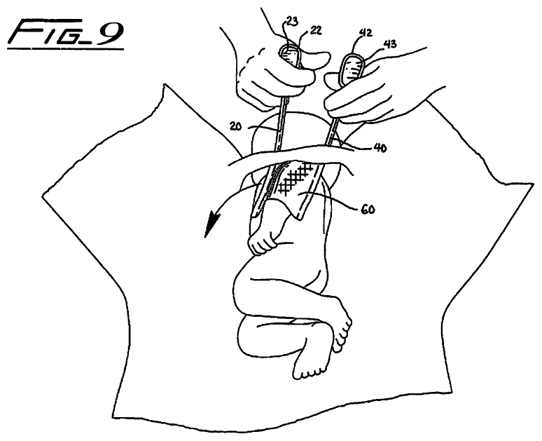

FIG. 8A illustrates an alternate embodiment wherein the engagement member 182 of device 180 is incorporated as part of a straight-line device similar to forceps. That is, device 180 has grasping handles 187, 188 which are pivotably attached at pivot pin 185. The handles extend into curved portions 184, 186 at distal ends which support the engagement member 182 therebetween. The looped grasping handles 187, 188 are opened and closed to move the engagement member 182 between spread and collapsed positions, respectively, or alternatively open and closed to move the engagement member 18 between collapsed and spread positions, respectively. The balloon(s), lubricant pump and other features of the various embodiments disclosed herein can be incorporated into the straight-line device of FIG. 8A.

In an alternate positioning/use of the device, shown in FIG. 8B, the arms 20, 40 of device 190 come in from below rather than above as in the foregoing embodiments. In this version, the physician would pull downward rather than push downwards to apply pressure on the fetal shoulder. The device 190 can be configured similar to device 10 of FIG. 1A or alternatively the arms and curves can be modified to facilitate the downward pull. A concavity can be provided on the surface of engagement member 60 contacting the body.

The engagement component 60 of the embodiments described herein can be attached to the arms 20, 40 in various ways, such as by suturing, screws, fasteners, etc.

The arms of the devices disclosed herein are preferably made of a rigid non bendable material, such as stainless steel, although other materials are also contemplated. The outer diameter can be constant or of varying diameters along various regions of its length. For example, the exposed linear portions 24 and 44 can have a larger diameter than the curved portions 28, 48 which are inserted into the body. By minimizing the size, a reduced profile is achieved to enable insertion into the limited space in the pelvis. In preferred embodiments, the outer diameter of the linear portions is about 3 mm, although other dimensions are also contemplated.

In use, if the baby's shoulder is engaged with the pubic arch of the mother and thereby blocking delivery of the baby in an incidence of shoulder dystocia, the device of the present invention is inserted with the arms and engagement component in the unopened (unspread) position such that the engagement section is collapsed or folded. The device is initially inserted along the side of the baby's head and along the baby's back. Then, with the arms still in the closed position, the device is maneuvered so the engagement section in the closed position rests against the baby's shoulder. (Alternatively, the device can be inserted by placing the arms under the pubic bone). Note the balloon can be inflated to act as a spacer in the embodiments incorporating an inflatable balloon(s). If a lubricant sac or container is incorporated into the device, it can be released prior to insertion of the device.

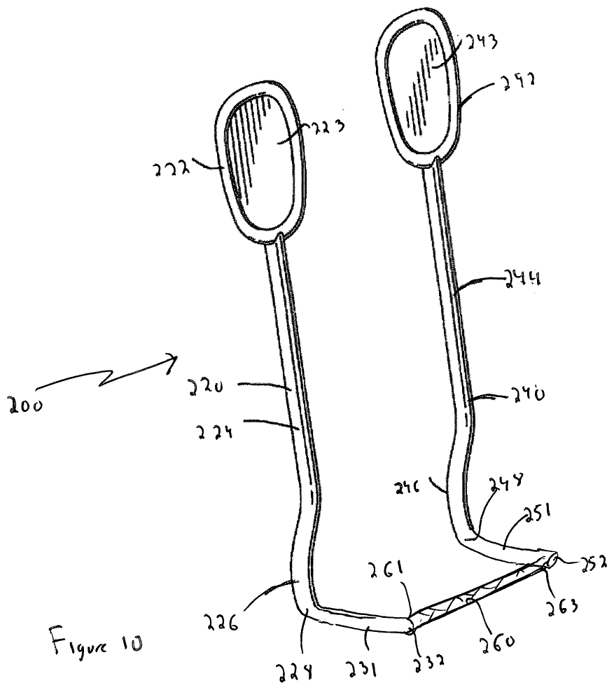

Next, the physician separates the grasping handles to spread the engagement section from the closed position to the open spread position thereby enveloping a portion of the baby's shoulder (see FIG. 9). Then, the physician presses down on the grasping handles utilizing the solid surfaces for leverage, to pivot the proximal portion of the arms downwardly to force engagement section downwardly. This force pushes the baby away from the pubic arch, thereby freeing the baby's shoulder from the pubic arch for delivery of the baby. Thus, the baby's shoulder is released from the arch in a quick and atraumatic fashion. (Note that in the embodiment of FIG. 8B, the physician would pull down once the engagement section is spread).

Note that in the devices incorporating a locking mechanism, the handles can be locked in various positions and at different times during use, e.g., in open and/or closed positions.

Note that in addition to the downward force, the handles can be rotated to the side (toward the fetal chest) to move the baby's shoulder to an oblique diameter of the pelvis.

In an alternative insertion method, the device can be inserted below the posterior shoulder of the baby and then manipulated to rotate the baby to an oblique diameter of the pelvis.

The engagement section or component can in some embodiments be substantially trapezoidal in shape having a proximal section with a width greater than the width of the distal section. In this manner, the varying tautness of the material of engagement section is “built into” the device. Note the material can be a cloth or any other suitable material(s) to provide sufficient force to move the baby's shoulder while being atraumatic to surrounding tissue. Due to the short width in the distal section, the material will be held more rigid than in the proximal section when the handles are moved apart. In one embodiment, the distal edge of the engagement section is about 3 cm and the proximal edge is about 6 cm, with the distance between the two edges being about 10 cm. Other dimensions are also contemplated. The two arms can be angled toward cach other to accommodate the substantially trapezoidal shaped material. During use of this configuration, the arms are manipulated away from each, i.e., toward a parallel position, thereby tightening the distal section. For example, in one embodiment, the distal edge can be stretched from about 3 cm to about 6 cm when the arms are moved to a substantially parallel position, with the proximal edge not changing width. This results in the proximal section remaining more flexible/stretchable to comfortably stretch over the baby's shoulder while the distal section is more rigid to apply sufficient force to move the baby. Note this substantially trapezoidal shape could also be used with the arms of any of the embodiments disclosed herein.

It should be noted that one or both arms can have a mechanism that will allow articulation with respect to one another and thus manipulation by the operator with one hand only.

### Alternative Embodiments Without Spread Engagement Member

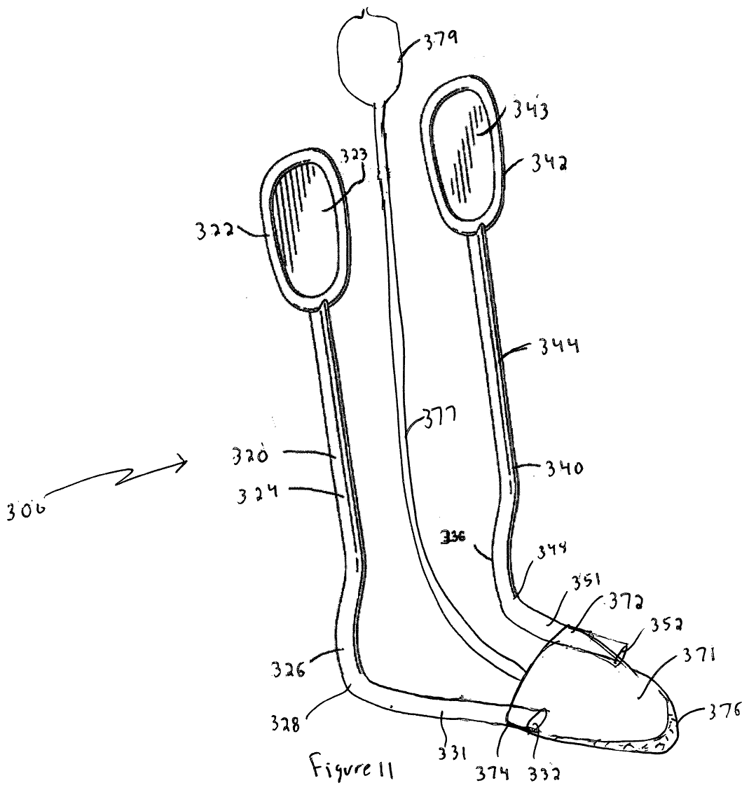

The devices discussed above provide improvements/enhancements to the inventor's prior device containing two arms supporting an engagement component/member therebetween which is spread by opening of the handles and capable of engaging a large surface area. The inventor also conceived of replacements for the engaging member which in certain applications could prove advantageous. These are disclosed in the embodiments of FIGS. 10-12. Note that the locking mechanisms and/or bag or sac disclosed above can be used with the devices of FIGS. 10-12 and the arms of these devices can have the alternative configurations disclosed herein, e.g., the configuration of FIGS. 1D and 1E.

Turning now to the first embodiment of these devices and with initial reference to FIG. 10, the device is designated generally by reference numeral 200 and includes a first arm 220, a second arm 240 and an elongated member 260 spanning the two arms 220, 240 at a distal region thereof. Arms 220 and 240 in a preferred embodiment are substantially identical, although they could be varied from each other as long as their function as described herein is maintained. Arm 220 has a grasping handle 222 at a proximal end, a substantially straight or substantially linear arm portion 224, a first curve 226, a curved portion 228, and a substantially straight or substantially linear arm portion 231 terminating in a distal tip 232. The arm 240 similarly has grasping handle 242 at a proximal end, a substantially straight or substantially linear arm portion 244, a first curve 246, a curved portion 248, and a substantially straight or substantially linear arm portion 251 terminating in a distal tip 252. The distal tips 232, 252 of arms 220, 240 are preferably round to provide an atraumatic blunt end. Alternatively, instead of linear arm portion 231, 251, the arms 231, 251 can curve upwardly as in the embodiment of FIG. 1C.

The arms can be composed of a metal or plastic material.

Note in an alternate embodiment, each arm 220, 240 can have a S-curve instead of the substantially linear portion 231, 251 as in FIG. 1D. This provides the arm with a double curve. Consequently, in this embodiment, the curve would conform to the contour of the baby's neck, and the lower aspect of the pubic bone. Consequently, in this embodiment, the curve would conform to the contour of the baby's neck, and the lower aspect of the pubic bone. Once in position, the arms would be opened in the same manner as in the embodiment of FIG. 10, and the handles manipulated in the same manner as in FIG. 10, to press the elongated member 260 against the baby's shoulder to provide a force in a direction away from the pubic arch to release the baby's shoulder.

The device of FIG. 10 can be inserted starting along the back of the baby and then moved to along the shoulder, or alternatively, due to the single curve, can be initially inserted over the fetal shoulder and under the pubic bone. This straighter arm version in some applications can be easier to slide into the pelvic area under the pubic arch. Also, this straighter version can better accommodate different pelvic shapes in certain applications.

The grasping handles 222 and 242 preferably have solid surfaces 223, 243, respectively, for engagement by the physician's thumbs to provide leverage for a downward movement of the device 200 as described below in conjunction with the method of use. The grasping handles 222 and 242 can alternatively be grasped by other fingers or held in the palm of the physician's hand as alternate ways to provide the appropriate leverage to apply the necessary force to dislodge the baby's shoulder from engagement with the pubic arch as described below. The grasping handles 222, 242 can be composed of a metal or plastic material.

The elongated member (component) of the device 200 is designated by reference numeral 260 and is in the form of a cord which spans a distal region between the two arms 220, 240 at the linear portions 231 and 251 (from end 261 to end 263). The cord 260 can alternatively be positioned in other regions along linear portions 231, 251. Diameters/widths of the cord 260 other than those shown can be utilized. The space between the two arms preferably ranges from about 4 cm to about 10 cm, and preferably between about 5 cm and about 8 cm, although other distances are also contemplated. The cord 260 likewise can have a similar length when stretched. When the handles 220, 240 are closer together, the cord 260 is not taut; when the handles 222, 242 are spread apart (see FIG. 10) to spread arms 220, 240, the cord 260 becomes taut so it can apply a force to the baby's shoulder (applied by downward force on the grasping handles in the same manner as described above) as it engages/contacts the shoulder.

Thus, the cord 260 forms an engagement member, but with a dimension much less than the engagement members of the embodiments of FIGS. 1-8. The cord 260 thus provides a smaller area for shoulder engagement which can be advantageous for access and use in certain clinical applications. More specifically, the cord 260, in order to move the shoulder from the pubic bone, puts pressure on the area between the shoulder joint and the neck, i.e., the cranial aspect of the shoulder, in comparison to the engagement member of the embodiments of FIGS. 1-8 which has a larger engagement surface, i.e., larger shoulder contact surface area. The cord 260 goes in a little bit so the clinician does not have to go all the way inside. For example, it can be placed on the portion of the shoulder that is visible. The cord can also be pushed against the shoulder and not over it as in the engagement member of the embodiments of FIGS. 1-8.

The cord 260 can be attached to the arms 220, 240 in various ways, such as by suturing, screws, fasteners, etc.

Note that instead of a cord, a string, a flat and thin connector or other type of elongated component could be utilized. They can be made of a synthetic material.

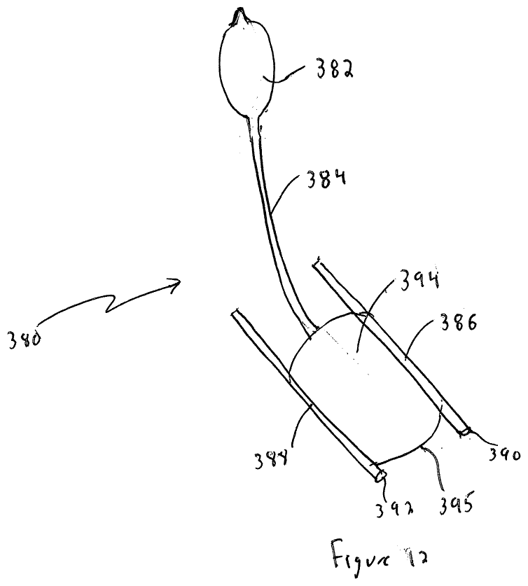

In the alternate embodiment of FIG. 11, instead of the engagement member of the embodiments of FIGS. 1-8, a balloon is provided which can slide under the pubic arch and over the shoulder in the collapsed state, and then inflated to apply a force to the shoulder. As shown, the balloon 371 of device 300 is supported by the linear sections 331, 351 of arms 320, 340, respectively. Arms 320 and 340 in a preferred embodiment are substantially identical, although they could be varied from each other as long as their function as described herein is maintained. Arm 320 has a grasping handle 322 at a proximal end, a substantially straight or substantially linear arm portion 324, a first curve 326 and a curved portion 328 transitioning into the substantially straight or substantially linear arm portion 331 terminating in a distal tip 332. The arm 340 similarly has grasping handle 342 at a proximal end, a substantially straight or substantially linear arm portion 344, a first curve 336 and a curved portion 348 transitioning into the substantially straight or substantially linear arm portion 351 terminating in a distal tip 352. The distal tips 332, 352 of arms 320, 340 are preferably round to provide an atraumatic blunt end. Alternatively, instead of linear arm portion 331, 351, the arms 331, 351 can curve upwardly as in FIG. 1C or have a S-curve as in FIG. 1D to provide the arm with a double curve conforming to the contour of the baby's neck and the lower aspect of the pubic bone. The arms can be composed of a metal or plastic material.

The balloon 371 can extend beyond the arms 20, 40 so the distal end 376 of balloon 371 is distal of the blunt ends 332, 352 of arms 320, 340. Alternatively, the balloon 371 can terminate flush with or terminate proximal of the blunt ends 332, 352. Although the arms 320, 340 are shown on the sides of the balloon, they can be in different positions other than those shown. For example, the arms 320, 340 can be below the balloon 371.

The balloon 371 is inflated by a pump 379 connected to the tube 377 which is fluidly connected to the balloon 371 via an opening in the distal end of the tube 377 communicating with an opening into the interior of the balloon 371. The pump 379 and tube 377 can be unattached to the handles 322, 342 as shown or alternatively the pump 379 can be attached to one of the handles 322, 324 or arms 320, 340, and/or the tube 377 can be attached to one of the handles 322, 324 or arms 320, 340. Such attachment would maintain the tube 377 and pump 379 in position so they would not need to be held by a clinician.

The balloon 371 forms an engagement member to contact/engage the baby's shoulder. In use, if the baby's shoulder is engaged with the pubic arch of the mother and thereby blocking delivery of the baby in an incidence of shoulder dystocia, the device 370 is inserted with the arms 320, 340 in a closed position and the balloon 371 in a deflated/collapsed position. The device is initially inserted along the side of the baby's head and along the baby's back. Then, with the arms 320, 340 still in the closed position, the device 370 is maneuvered so the balloon 371 in the collapsed position rests against the baby's shoulder. (Alternatively, the device can be inserted by placing the arms 320, 340 under the pubic bone). The balloon 371 is then inflated, acting as a spacer, and then applying a force against the baby's shoulder as the physician presses down on the grasping handles 323, 343 utilizing the solid surfaces for leverage, to pivot the proximal portion of the arms 320, 340 downwardly to force the balloon 371 downwardly. This force pushes the baby away from the pubic arch, thereby freeing the baby's shoulder from the pubic arch for delivery of the baby. Thus, the baby's shoulder is released from the arch in a quick and atraumatic fashion.

In the alternate embodiment of FIG. 12, the balloon 394 of device 380 is supported on arms 386 and 388. Arms 386, 388 terminate in blunt ends 390, 392. The arms 386, 388 can be linear or can have a curved surface. The balloon 394 can terminate at region 395 proximal of the ends 390, 392; alternatively, the balloon 394 can terminate flush with or distal of ends 390, 392. The balloon 394 is inflated via pump 382 which fluidly communicates with an interior of balloon 394 via tube 384. The balloon 394 in the collapsed position is slid under the pubic arch and over the shoulder and then inflated. The balloon 394 is shown in the deflated/non-inflated state/condition in FIG. 12. Inflation of balloon 394 applies a force to the baby's shoulder.

Note, in use, inflation of the balloon 371 or 394 itself can apply pressure to release the baby's shoulder. Alternatively, or in addition, the arms of the device 370 or 380 can be used to apply a downward pressure on the balloon to release the shoulder in a manner similar to engagement member 60 of FIG. 1.

The balloon 371 or 394 can have separate/independent inflatable compartments to enable inflation of select regions to engage the baby's shoulder. A single pump or multiple pumps can be used to inflate the separate compartments.

The balloon 371 or 394 can be inflated to various pressure/sizes as selected by the user via the hand pump. Automatic pumps for inflating the balloons are also contemplated.

The balloon 371, 394 in some embodiments has a more flexible proximal section to provide greater elasticity. The distal section can more rigid to apply a sufficient force against the baby's shoulder when a downward force is applied by manipulation of the arms.

In some embodiments, the handles of the device 200 or 300 are configured to lock (interlock) together. That is, a locking mechanism can be utilized to retain the handles in position with respect to each other during use. In use, the physician separates the grasping handles to spread them from the closed position to the open spread position. The handles can be locked in the open position and/or the closed position. In some embodiments, the locking mechanism enables locking of the handles in various positions with respect to each other. The locking of the handles facilitates the use/manipulation of the device when applying downward pressure as the handles are not movable relative to each other. The locking devices can include those described above, as well as other alternatives.

Common features, alternatives and advantages of the devices of FIGS. 1-12 will now be discussed.

In certain applications, in addition to the downward force, the handles of the devices in the embodiments disclosed herein can be rotated to the side (toward the fetal chest) to move the baby's shoulder to an oblique diameter of the pelvis. That is, such pivoting movement of the handles can rotate the baby, e.g., rotate the baby's shoulder, about 45 degrees, gaining two advantages: 1) the anterior shoulder is not hindered by the pubic arch; and 2) the oblique diameter of the pelvic outlet is larger than the anterior-posterior diameter of the mother, and therefore it is easier for the baby to come out through this position aided by pushing of the mother. The locking of the handles can facilitate such rotational/pivoting movement of the device as the handles are not movable relative to each other.

In some embodiments, the front of one or both of the arms could have multiple protrusions and the back of one or both arms have multiple depressions so that when the arms are crossed and pressed together, they interlock due to the protrusion/depression engagement.

It should be appreciated that the handle locking mechanisms can be used with any of the embodiments of the device disclosed herein.

The arms of any of the devices disclosed herein are preferably made of a rigid non bendable material, such as stainless steel, although other materials are also contemplated. The outer diameter can be constant or of varying diameters along various regions of its length. For example, the exposed linear portions 24, 44 or 224 and 244 can have a larger diameter than the curved portions 28, 48 or 228, 248 which are inserted into the body. By minimizing the size, a reduced profile is achieved to enable insertion into the limited space in the pelvis. In preferred embodiments, the outer diameter of the linear portions is about 3 mm, although other dimensions are also contemplated.

In use, if the baby's shoulder is engaged with the pubic arch of the mother and thereby blocking delivery of the baby in an incidence of shoulder dystocia, the device of the present invention is inserted with the arms in the unopened (unspread) position such that the engagement section is not taut (FIG. 1 embodiment) or the balloon not inflated (FIG. 11 embodiment) or the cord not taut (FIG. 10 embodiment), etc. The device is initially inserted along the side of the baby's head and along the baby's back or directly under the pubic bone. Then, with the arms still in the closed position, the device is maneuvered so the engagement member, cord or uninflated balloon (depending on the embodiment) rests against the baby's shoulder. Note the arms can be inserted without being approximated together.

Next, the physician separates the grasping handles which depending on the embodiment, spreads the engagement member or cord to make it taut or inflates the balloon in the balloon embodiment. Then, the physician presses down on the grasping handles utilizing the solid surfaces for leverage, to pivot the proximal portion of the arms downwardly to force the engagement member, balloon on cord downwardly. This force pushes the baby away from the pubic arch, thereby freeing the baby's shoulder from the pubic arch for delivery of the baby. Thus, the baby's shoulder is released from the arch in a quick and atraumatic fashion. In the embodiments utilizing the balloon, the inflated balloon provides the releasing force against the shoulder via manipulation of the handles.

Note that in the devices incorporating a locking mechanism, the handles can be locked in various positions and at different times during use, e.g., in open, partially open, and/or closed positions.

Note that in addition to the downward force, the handles can be rotated to the side (toward the fetal chest) or back to move the baby's shoulder to an oblique diameter of the pelvis.

In an alternative insertion method, any of the devices herein can be inserted below the posterior shoulder of the baby and then manipulated to rotate the baby to an oblique diameter of the pelvis.

In an alternate insertion method, any of the devices herein can be inserted from below rather than above for pulling downward as described above in conjunction with FIG. 8B.

It should be noted that one or both arms of any of the devices disclosed herein can have a mechanism that will allow articulation with respect to one another and thus manipulation by the operator with one hand only. It should be noted that with the devices made of a balloon and a pump without handles, the inflated balloon can be placed in the pelvis with the operator's fingers.

The devices disclosed herein have several qualities that allow it to resolve the shoulder dystocia quickly and safely:
1. A unique structure that allows it to negotiate the tight “S” shaped contact area between the fetal shoulder and neck and the aspects of the maternal pubic arch.
2. Ability to provide pressure over large areas of the fetal body (in the embodiments utilizing the spread out engagement member or in some instances the balloon, depending on tis configuration), specifically to the cranial aspect of the humerus and the shoulder joint, thus avoiding pressure points and minimizing the risk of fractures or soft tissue injuries.
3. The device does not apply pressure to the maternal tissues, and does not use parts of the maternal anatomy as fulcrum points, thus minimizing the risk of maternal injury.
4. The device allows the operator a good grip of the fetal shoulder girdle, thus allowing the operator to rotate the fetal torso with ease.
5. The application of the device uses both of the operator's hands (in the two handle versions), each one manipulating another arm of the device. Thus, it prevents the instinctive traction that the operator wants to apply to the fetal head until after the fetal shoulder is removed from behind the pubic arch. That is, under the emergency conditions of shoulder dystocia, health providers might underestimate the force that they apply to the fetal head and the intensity of the traction can injure the fetus, especially through damage to the brachial plexus.

The devices disclosed herein utilizing the spread engagement member of FIGS. 1A-8B further have one or more enhancements to help resolve shoulder dystocia:
a) Inflatable spacers to engage select anatomic structures
b) Concavities to improve conformance to the baby's anatomy
c) Built in lubricant delivery
d) Locking mechanism to interlock handles
e) Additional handle structures for grasping and using the device to apply traction and maneuver the baby

The devices of FIGS. 10-12 which are alternatives to the spread engagement member further have one or more variations to help resolve shoulder dystocia:
a) Inflatable balloons to engage the shoulder
b) Reduced shoulder surface contact area via a cord or string
c) Locking mechanism to interlock handles

Any of the devices disclosed herein can be composed of disposable materials or reusable sterilizable materials.

In some embodiments, the device can be inserted below the posterior shoulder of the baby to allow rotation of the baby.

Note the devices disclosed herein are not limited for use in shoulder dystocia but could also be used in other clinical situations requiring maneuvering of the fetal shoulder to enable delivery.

The present invention also provides methods for treating shoulder dystocia including the steps of providing a device having first and second arms and an engagement section extending between the arms, inserting the device into a birth canal adjacent the baby, and manipulating the first and second arms so the engagement section or contact section applies a force on the baby to free a shoulder of the baby from the pubic arch for delivery. The engagement or contact member can include the spreadable material of FIGS. 1A-8B, the cord of FIG. 10 or the balloon of FIG. 11. The method can further include providing a lubricant to the engagement member or contact member. The method can also include the step of locking the first and second arms of the device in the open/spread position.

Note the devices of the present invention can be used by a physician, clinician, e.g., nurse, midwife or other birth attendant or health care professional.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure as defined by the claims appended hereto.

Although the apparatus and methods of the subject invention have been described with respect to preferred embodiments, those skilled in the art will readily appreciate that changes and modifications may be made thereto without departing from the spirit and scope of the present invention as defined by the appended claims.

It will be understood by those skilled in the art that the above particular embodiments are shown and described by way of illustration only. The principles and the features of the present disclosure may be employed in various and numerous embodiments thereof without departing from the scope and spirit of the invention as claimed.

Additionally, persons skilled in the art will understand that the elements and features shown or described in connection with one embodiment may be combined with those of another embodiment without departing from the scope of the present invention and will appreciate further features and advantages of the presently disclosed subject matter based on the description provided.

Throughout the present disclosure, terms such as “approximately,” “about”, “generally,” “substantially,” and the like should be understood to allow for variations in any numerical range or concept with which they are associated. It is intended that the use of terms such as “approximately”, “about”, “substantially”, and “generally” should be understood to encompass variations on the order of 25%, or to allow for manufacturing tolerances and/or deviations in design.

The recitation of numerical ranges by endpoints includes all numbers within the range.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present invention.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

1. A medical device for treating shoulder dystocia comprising:
a first arm having a proximal portion and a distal portion, the first arm having a first curved surface;
a second arm having a proximal portion and a distal portion, the second arm having a second curved surface;
a shoulder engagement section positioned at least between the distal portion of the first and second arms and connecting the first and second arms, the engagement section having an upper surface and a lower surface, the lower surface having an atraumatic surface and configured to contact and press down on a baby to reposition the baby upon manipulation of the first and second arms; and
a spacer extending from the upper surface of the engagement section.

2. The medical device of claim 1, wherein the engagement section comprises a first section and a second section, wherein the second section is more flexible than the first section, the second section positioned proximal of the first section.

3. The medical device of claim 1, wherein the spacer comprises a first inflatable balloon.

4. The medical device of claim 3, further comprising a pump and a tube fluidly connected to the first balloon, the pump attached to one of more of the first arm, second arm or engagement member.

5. The medical device of claim 3, wherein the spacer comprises a second balloon positioned adjacent the first balloon.

6. The medical device of claim 5, wherein the first and second balloons are independently inflatable.

7. The medical device of claim 3, wherein the first balloon has a first compartment and a second separate compartment, the first and second compartments independently inflatable to selectively space regions of the engagement member.

8. A medical device for treating shoulder dystocia comprising:
a first arm having a proximal portion and a distal portion, the first arm having a first curved surface;
a second arm having a proximal portion and a distal portion, the second arm having a second curved surface;
a shoulder engagement section positioned at least between the distal portion of the first and second arms and connecting the first and second arms, the engagement section having an upper surface and a lower surface, the lower surface having an atraumatic surface and configured to contact and press down on a baby to reposition the baby upon manipulation of the first and second arms, the upper surface having a concave section forming a depression to accommodate the shoulder of the baby.

9. The medical device of claim 8, wherein the concave section is formed in a central region of the engagement member.

10. The medical device of claim 8, wherein a second concave section is positioned in the upper surface of the engagement member.

11. The medical device of claim 8, further comprising a concave section on the lower surface of the engagement member.

12-16. (canceled)

17. A medical device for treating shoulder dystocia comprising:
a first arm having a proximal portion and a distal portion, the first arm having a first curved surface;
a second arm having a proximal portion and a distal portion, the second arm having a second curved surface;
a shoulder engagement section positioned at least between the distal portion of the first and second arms and connecting the first and second arms, the engagement section having an upper surface and a lower surface, the lower surface having an atraumatic surface and configured to contact and press down on a baby to reposition the baby upon manipulation of the first and second arms; and
a lubricant dispersible on the engagement member.

18. The medical device of claim 17, wherein the lubricant is contained in a lubricant bag positioned adjacent the engagement section.

19. The medical device of claim 17, wherein the lubricant encapsulates the engagement section.

20. The medical device of claim 18, wherein the lubricant bag is a tear away bag openable to release the lubricant on the engagement section.

21. The medical device of claim 17, wherein the lubricant is contained in a container, the container connected to a tube having an opening adjacent the engagement section.

22. The medical device of claim 17, wherein the lubricant is contained in a container, the container connected to a tube, wherein the tube is divided into a first and second tube section, the first tube section having a first opening and the second tube section having a second opening spaced from the first tube opening.

23. The medical device of claim 17, wherein the engagement section is one of a spreadable material, balloon or cord.

24-32. (canceled)

33. The medical device of claim 8, further comprising a lubricant on the engagement member.

34. The medical device of claim 33, wherein the lubricant is contained in a lubricant bag positioned adjacent the engagement section.
